(19) United States
(12) Patent Application Publication
(10) Pub. No.: US 2025/0163521 A1
(43) Pub. Date: May 22, 2025

(54) PRIMER SET, REAGENT COMPOSITION AND METHOD FOR THE DETECTION OF NEISSERIA MENINGITIDIS

(71) Applicant: GENOMTEC S.A., Wroclaw (PL)

(72) Inventors: Miron TOKARSKI, Brzeg (PL); Malgorzata MALODOBRA-MAZUR, Wroclaw (PL); lzabela PIELKA, Konopiska (PL)

(21) Appl. No.: 18/577,040

(22) PCT Filed: Jul. 7, 2022
(86) PCT No.: PCT/PL2022/050044

§ 371 Date: Jan. 5, 2024

(30) Foreign Application Priority Data
Jul. 7, 2021 (PL) P.438373

(51) Int. Cl. C12Q 1/689; C12Q 1/6851

(52) U.S. Cl. CPC C12Q 1/689; C12Q 1/6851; C12Q 2600/166

(57) ABSTRACT

The first subject of the invention is a set of primers for amplifying the nucleotide sequence of the dem gene of Neisseria meningitidis bacteria. The second subject of the invention is a method for detecting Neisseria meningitidis bacteria. Another subject of the invention is a method of detecting an infection caused by Neisseria meningitidis bacteria. The fourth subject of the invention is a kit for detecting an infection caused by Neisseria meningitidis bacteria.

### CROSS-REFERENCE TO RELATED APPLICATIONS

This application is the U.S. Natl. Stage of International Appln. PCT/PL2022/050044, filed on Jul. 7, 2022, which itself claims the benefit of Polish Appln. P438373, filed on Jul. 7, 2021.

### REFERENCE TO SEQUENCE LISTING SUBMITTED ELECTRONICALLY

The instant application contains a Sequence Listing which has been submitted electronically in XML format and is hereby incorporated by reference in its entirety. Said XML copy, created on Nov. 12, 2024, is named SEQLXML26.xml and is 16, 384 bytes in size.

### FIELD OF THE INVENTION

The invention relates to a set of primers for detecting Neisseria meningitidis (NM) bacteria, a method for detecting Neisseria meningitidis using the set of primers, and the use of the set of primers for detecting Neisseria meningitidis bacteria. The invention is applicable in medical diagnostics.

### BACKGROUND OF THE INVENTION

Neisseria meningitidis is a gram-negative bacterium. It has the form of diplococcus of an aerobic nature. The bacterium has the ability to grow on a variety of agar media, including blood or trypticase-soy agar, chocolate agar, or Mueller-Hinton agar. At least 13 different groups belonging to Neisseria meningitidis, constituting serogroups, are known. Six serogroups are pathogenic to humans, i.e. A, B, C, W-135, X and Y, causing life-threatening diseases including meningitis and sepsis, a life-threatening organ dysfunction caused by deregulated systemic response to infection.

It is estimated that approximately 1.2 million cases of Neisseria meningitidis infections are diagnosed worldwide each year, with mortality rate of approximately 135,000 deaths annually. Moreover, infections caused by Neisseria meningitidis are characterized by a relatively high risk of numerous complications, such as hearing loss, cognitive dysfunctions or muscle and nervous motor activity disorders.

Due to the threat to life posed by infection with the Neisseria meningitidis, it is important that the diagnosis is performed quickly and that the diagnostic methods are characterized by high sensitivity and specificity.

Laboratory diagnostics of Neisseria meningitidis is based primarily on detecting bacteria in nasopharyngeal swabs (the most common habitat in carriers) and in blood or cerebrospinal fluid collected form patients showing symptoms of systemic encephalitis. Possible infection or methods of detecting Neisseria meningitidis are bacterial culture or genetic testing, including the most commonly used Real-Time PCR. Assays based on bacterial culture of Neisseria meningitidis bacteria, despite their high sensitivity and specificity, are labour-intensive and time-consuming, on average requiring from 24 h to 48 h to confirm the infection.

The methods characterized by the greatest specificity and sensitivity are those involving the detection of Neisseria meningitidis nucleic acids in biological sample (the so-called NAAT methods—Nucleic Acid Amplification Tests). The most commonly used NAAT tests are assays based on Real-Time PCR method. Many different tests using the Real-Time PCR technique are available on the market, but despite the fierce competition, these methods are still relatively expensive. Moreover, they require highly specialized personnel, expensive devices, and the isolation of genetic material from the patient's sample is necessary. Furthermore, since cyclic heating and cooling of the reaction mixture is necessary, this method is relatively long, and the devices used consume relatively large amounts of energy for the diagnostic process.

Isothermal methods, including LAMP (Loop-mediated isothermal amplification) technique, are methods that allow to accelerate the diagnostic process and reduce the cost of energy and reagents needed to perform the analysis. Moreover, according to the literature data, these methods are characterized by higher sensitivity and specificity than the aforementioned Real-Time PCR technique, they are also much faster. Their isothermal course does not require specialized equipment.

Due to the low equipment requirements, isothermal methods are an ideal diagnostic solution for primary care units (POCT—point-of-care testing), where the test can be performed in the general practitioners' or specialist doctor during the first contact of a patient with the doctor. This solution allows for a quick diagnostic test (in no more than 15 minutes), which allows for selection of a targeted therapy during the very first visit. This is especially important in the case of infection with the Neisseria meningitidis bacterium due to the rapid development of the infection, which in a short time leads to an immediately life-threatening condition. Delayed diagnosis of NM infection increases the patient's risk of death, as well as the risk of developing serious complications. Moreover, prompt diagnosis and early treatment initiation increase the chances of survival and minimize the risk of long-term complications. On the other hand, the use of freeze-dried reagents allows the tests to be stored at room temperature, without the need to freeze the diagnostic assays.

The use of primers in the LAMP method for the diagnosis of Neisseria meningitidis is known from the patent applications published so far: CN110656192A; US20130252248A1; WO2014077417A1. The LAMP method is disclosed, for example, in patent specifications WO0028082, WO0224902. In the above-mentioned patent disclosures, the detection limit of the methods is much higher and about 100 copies. Some patent applications are based on end-point detection with detection of the end product of the reaction using agarose gel electrophoresis or by turbidimetric measurement. Moreover, in the described patent applications, the analysis time and waiting for a positive result is about 60 minutes. Besides, most of the kits developed and described above are not applicable in POCT diagnostics, and their main application is in laboratories.

Therefore, there is still a need to provide a diagnostic method using appropriately refined sets of primers used for the diagnosis of Neisseria meningitidis (all pathogenic serotypes) with the LAMP method, intended for use in point-of-care testing, which allows the detection of bacteria with a very low detection limit (≥5 copies/reaction) in a short time (≤15 min). Unexpectedly, the above-mentioned problem was solved by the present invention. The use of fluorescent markers allowed for a significant reduction of the detection limit (≥5 copies). In addition, fluorescent dyes allow for detecting of the reaction product in the Real-Time technology, which significantly shortens the reaction time (≤15 min) and enables the quantitative measurement of the pathogen.

### DETAILED DESCRIPTION OF THE INVENTION

The first subject of the invention is a set of primers for amplifying the nucleotide sequence of the FrpA (iron-regulated protein) gene of Neisseria meningitidis bacteria, characterized in that it comprises a set of internal primers with the following nucleotide sequences a) and b), as well as a set of external primers containing the following nucleotide sequences c) and d) specific for a selected fragment the iron-regulated protein (FrpA) gene of Neisseria meningitidis bacteria:
a) 5′ CGAGCGTATCATTGCCATTGCC 3′-(nucleic sequence SEQ ID NO: 3 or its reverse and complementary sequence), linked from the 3′ end, preferably by TTTT bridge, to the sequence 5′ CGGGGATGACCTGCTGAA 3′-(nucleic sequence SEQ ID NO: 4 or its reverse and complementary sequence)
b) 5′ ACGACGCCCTGTACGGCTATA 3′-(nucleic sequence SEQ ID NO: 5 or its reverse and complementary sequence), linked at the 3′ end, preferably by TTTT bridge, to the sequence 5′ TACCGTCTTCGCCGTTCAA 3′-(nucleic sequence SEQ ID NO: 6 or its reverse and complementary sequence)
c) 5′ GGGCGATGACTATCTGTACG 3′ nucleic sequence SEQ ID NO: 1 or its reverse and complementary sequence, and
d) 5′ CACCGCCGATTAGAGTGTC 3′ nucleic sequence SEQ ID NO: 2 or its reverse and complementary sequence.

In a preferred embodiment of the invention, the primer set comprises a set of loop primer sequences comprising nucleic sequences contained in or complementary to the Neisseria meningitidis FrpA gene SEQ ID NO: 7-5′ TACTGTCGTTGCCTGCATCACC 3′ and SEQ ID NO: 8: 5′ GGTAACGATGTACTGAATGGTGG 3′ or sequences reverse and complementary thereof.

The second subject of the invention is a method for detecting Neisseria meningitidis bacteria, characterized in that a selected region of the nucleotide sequence of the Neisseria meningitidis genome (iron-regulated protein FrpA gene fragment) is amplified using a primer set as defined in the first subject of the invention and with the amplification method being the LAMP method.

In a preferred embodiment, the amplification is carried out with a temperature profile: 68° C., 40 min.

In a further preferred embodiment of the invention, the end-point reaction is carried out with a temperature profile of 80° C., 5 min. performed after the amplification step.

The third subject of the invention is a method for detecting an infection caused by the Neisseria meningitidis bacterium, characterized in that it comprises the detection method defined in the second subject of the invention.

The fourth subject of the invention is a kit for detecting an infection caused by Neisseria meningitidis bacteria, characterized in that it comprises a set of primers as defined in the first subject of the invention.

In a preferred embodiment of the invention, the infection detection kit comprises 5.0 μl WarmStart LAMP Master Mix.

In a further preferred embodiment of the invention, individual amplification primers as defined in the first subject of the invention are utilised, with the primers having the following concentrations: 0.13 μM F3, 0.13 μM B3, 1.06 μM FIP, 1.06 μM BIP, 0.26 μM LoopF, 0.26 μM LoopB; together with D-(+)-Trehalose dihydrate-6%; mannitol-1.25%; fluorescent marker interacting with double-stranded DNA—EvaGreen≤1× (Biotium) or Fluorescent Dye (New England Biolabs) in the amount of ≤1 μl or Syto-13≤16 μM (ThermoFisher Scientific) or SYTO-82≤16 μM (ThermoFisher Scientific) or another fluorescent dye interacting with double-stranded DNA at a concentration that does not inhibit the amplification reaction.

The advantage of the primer sets of the invention for detecting Neisseria meningitidis, as well as the method for detecting Neisseria meningitidis infection and the method of detecting the amplification products is the possibility of using them in medical diagnostics at the point of care (POCT) in the target application with a portable genetic analyser. Freeze-drying of the reaction mixtures of the invention allows the diagnostic kits to be stored at room temperature without reducing the diagnostic parameters of the assays. Furthermore, the use of a fluorescent dye to detect the amplification product increases the sensitivity of the method and allows to lower the detection limit (down to 5 genome copies/reaction), as well as it enables the quantitative measurement of the bacteria in the test sample.

### BRIEF DESCRIPTION OF THE DRAWINGS

Exemplary embodiments of the invention are presented in the drawing, in which

FIG. 1 shows the sensitivity characteristics of the method, where a specific signal was obtained with the template: Neisseria meningitidis Quantitative DNA (ATCC® 700532DQ™) over the range of 1000-5 copies/μl, but there was no product in NTC, FIG. 1: lane 1: mass marker (Quick-Load® Purple 100 bp DNA Ladder, NewEngland Biolabs); lane 2: 1000 copies of NM; lane 3: 100 copies of NM; lane 4: 50 copies of NM; lane 5: 25 copies of NM; lane 6: 20 copies of NM; lane 7: 10 copies of NM; lane 8: 5 copies of NM; lane 9: NTC;

FIG. 2 shows the sensitivity of the method of the invention as measured by testing a serial dilution of the Neisseria meningitidis Quantitative DNA (ATCC® 700532DQ™) a range standard over of 1000-5 copies/reaction of the DNA standard, where the product amplification was measured in real time. The results of the real-time Neisseria meningitidis detection are presented in Table 1, giving the minimum time required to detect the fluorescence signal;

FIG. 3 shows the specificity of the product obtained after Neisseria meningitidis detection as measured by the dissociation curve of the amplification product using the Neisseria meningitidis Quantitative DNA (ATCC® 700532DQ™) standard over the range 1000-5 copies/reaction by real-time fluorescence measurement, with a target dissociation temperature (Tm) of 88.5° C. for a specific reaction product;

FIGS. 4 and 5 show the specificity of the method of the invention with standard matrices of a number of pathogens potentially present in the tested biological material as natural physiological flora, those which may result from co-infections or those which share similar genomic sequences; FIG. 4: lane 1: mass marker (Quick-Load® Purple 100 bp DNA Ladder, NewEngland Biolabs); lanes 2 and 3: methicillin-sensitive Staphylococcus aureus (MSSA); lanes 4 and 5: Pseudomonas aeruginosa; lanes 6 and 7: Borrelia afzelii; lanes 8 and 9: Influenza B virus; lanes 10 and 11: Moraxella catarrhalis; lanes 12 and 13: Campylobacter jejuni; lanes 14 and 15: Influenza A H1N1; lanes 16 and 17: Acinetobacter baumannii; lanes 18 and 19: HBV; lanes 20 and 21: Influenza A H3N2; lanes 22 and 23: Listeria monocytogenes; lanes 24 and 25: HHV-1; lanes 26 and 27: Borrelia burgdorferi; lanes 28 and 29: Legionella pneumoniae; lanes 30 and 31: Homo sapiens; lanes 32 and 33: Klebsiella pneumoniae; lanes 34 and 35: Haemophilus ducreyi; lanes 36 and 37: Bordetella pertussis; lanes 38 and 39: HHV-5; lanes 40 and 41: Neisseria gonorrhoeae; lanes 42 and 43: Lactobacillus gasseri; lanes 44, 45: Streptococcus pyogenes; 46, 47: Lactobacillus jensenii; 48, 49: methicillin-resistant Staphylococcus aureus (MRSA); 50, 51: Bacteroides fragilis; 52, 53: Enterococcus faecalis; 54, 55: Escherichia coli; 56, 57: Enterococcus faecium; 58, 59: Mobiluncus mulieris; FIG. 5, lane 1: mass marker (Quick-Load® Purple 100 bp DNA Ladder, NewEngland Biolabs); lanes 2 and 3: Neisseria meningitidis; lanes 4, 5: NTC; in turn, FIG. 6 shows the specificity of the method in relation to other serogroups pathogenic to humans, i.e. A, C and E. The method detects all tested serogroups that are a causal factor of infectious diseases caused by N. meningitidis, including those with a particularly life-threatening course;

FIG. 6: lane 1: mass marker (Quick-Load® Purple 100 bp DNA Ladder, NewEngland Biolabs); lanes 2 and 3: Neisseria meningitidis serogroup C; lanes 4 and 5: Neisseria meningitidis serogroup E; lanes 6 and 7: Neisseria meningitidis serogroup A; lanes 8 and 9: Neisseria meningitidis serogroup C; lanes 10 and 11: NTC.

### EXAMPLES

### Example 1. Primer Sequences

The sequences of specific oligonucleotides used for the detection of Neisseria meningitidis genetic material using LAMP technology are presented and characterized below.

1. The NM FrpAF3 oligonucleotide sequence: 5′ GGGCGATGACTATCTGTACG 3′ is a sequence identical to the Neisseria meningitidis FrpA gene (5′-3′ strand).

2. The NM FrpAB3 oligonucleotide sequence: 5′ CACCGCCGATTAGAGTGTC 3′ is a complementary fragment of the Neisseria meningitidis FrpA gene (5′-3′ strand) 176 nucleotides away from the 3′ end of the oligonucleotide 1.

3. The NM Frp1F2 oligonucleotide sequence: 5′ CGGGGATGACCTGCTGAA 3′ is a sequence identical to the Neisseria meningitidis FrpA gene (5′-3′ strand) 7 nucleotides away from the 3′ end of the oligonucleotide 1.

4. The NM FrpAB2 oligonucleotide sequence: 5′ TACCGTCTTCGCCGTTCAA 3′ is a complementary fragment of the Neisseria meningitidis FrpA gene (5′-3′ strand) 155 nucleotides away from the 3′ end of the oligonucleotide 1.

5. The NM FrpAF1c oligonucleotide sequence: 5′ CGAGCGTATCATTGCCATTGCC 3′ is a complementary fragment of the Neisseria meningitidis FrpA gene (5′-3′ strand) 56 nucleotides away from the 3′ end of the oligonucleotide 1.

6. The NM FrpAB1c oligonucleotide sequence: 5′ ACGACGCCCTGTACGGCTATA 3′ is a sequence identical to the Neisseria meningitidis FrpA gene (5′-3′ strand) 93 nucleotides away from the 3′ end of the oligonucleotide 1.

7. The NM FrpALoopF oligonucleotide sequence: 5′ TACTGTCGTTGCCTGCATCACC 3′.

8. The NM FrpALoopB oligonucleotide sequence: 5′ GGTAACGATGTACTGAATGGTGG 3′.

The sequences of the F1c and F2 oligonucleotides have preferably been linked by a TTTT bridge and used as FIP. The sequences of the B1c and B2 oligonucleotides have preferably been linked by a TTTT bridge and used as BIP.

### Example 2

The method of amplifying the FrpA gene of Neisseria meningitidis using the oligonucleotides characterized in Example 1 with LAMP technology and the following composition of the reaction mixture:

5.0 μl WarmStart LAMP 2X Master Mix
0.13 μM F3
0.13 μM B3
1.06 μM FIP
1.06 μM BIP
0.26 μM LoopF
0.26 μM LoopB
D-(+)-Trehalose dihydrate - 6%
Mannitol - 1.25%

Fluorescent marker interacting with double-stranded DNA-EvaGreen (Biotium) ≤1× or Fluorescent dye 50× (New England Biolabs) in the amount of ≤1 μl or GreenFluorescent Dye (Lucigen) in the amount of ≤1 μl or Syto-13≤16 μM or SYTO-82≤16 μM or another fluorescent dye that interacts with double-stranded DNA at a concentration that does not inhibit the amplification reaction.
DNA template ≥5 copies/reaction

Total reaction volume adjusted to 10 μl with DNase and RNase free water.

### Example 3

The method of amplifying the FrpA gene of Neisseria meningitidis using the oligonucleotides characterized in Example 1 and Example 2 with LAMP technology and the composition of the reaction mixture characterized in Example 3 with the following temperature profile:
1) 68° C., 40 min
2) preferably for end-point reactions 80° C., 5 min.

### Example 4

The method of amplification and detection of the FrpA gene of Neisseria meningitidis using the oligonucleotides characterized in Example 1 and Example 2 with LAMP technology and the composition of the reaction mixture characterized in Example 2 with the temperature profile characterized in Example 3 and the detection method described below.

A fluorescent dye, capable of interacting with double-stranded DNA is used and added to the reaction mixture in an amount of 0.5 μl EvaGreen 20×; 0.5 μl or a concentration of ≤1×; ≤16 μM for GreenFluorescent Dye (Lucigen); SYTO-13 and SYTO-82, respectively, before starting the reaction, real-time and/or end-point measurement. Excitation wavelength in the range similar to the FAM dye-490-500 nm (optimally 494 nm) for EvaGreen; Fluorescent dye 50× (New England Biolabs), GreenFluorescent Dye (Lucigen); SYTO-13 dyes and 535 nm (optimally 541 nm) for the SYTO-82 dye; emission wavelength in the range of 509-530 nm (optimally 518 nm) for EvaGreen; GreenFluorescent Dye (Lucigen); SYTO-13 dyes and 556 nm (optimally 560 nm) for the SYTO-82 dye, the method of detection, change recording time starting from 15 minutes from the start of the reaction for Neisseria meningitidis and the negative control.

### Example 5

The method of preparation and freeze-drying of reagents for detecting the amplification and detection of the Neisseria meningitidis FrpA gene using the oligonucleotides characterized in Example 1 and Example 2 with LAMP technology and the composition of the reaction mixture characterized in Example 2 with the temperature profile characterized in Example 3 and the detection method described in Example 4.

### Example 6. Description of the Freeze-Drying Process

The reaction components were mixed according to the composition described in Example 2, except the template DNA, to a total volume of 10 μl. The mixture was transferred to 0.2 ml tubes and subjected to the freeze-drying process according to the parameters below.

The mixture placed in test tubes was pre-cooled to −80° C. for 2 hours. Then the freeze-drying process was carried out at the temperature of −80° C. for 3 hours under the pressure of 5⁻² mBar.

### Example 7. Sensitivity of the Method

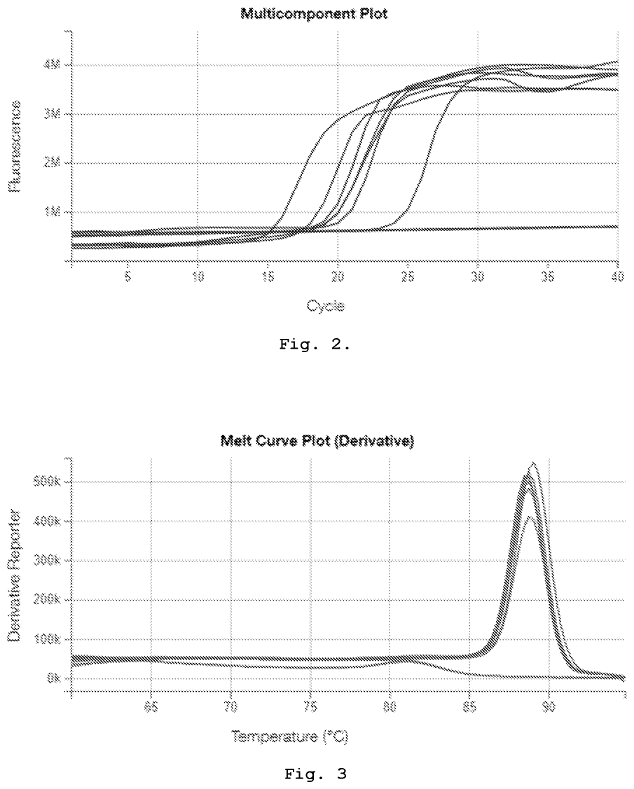

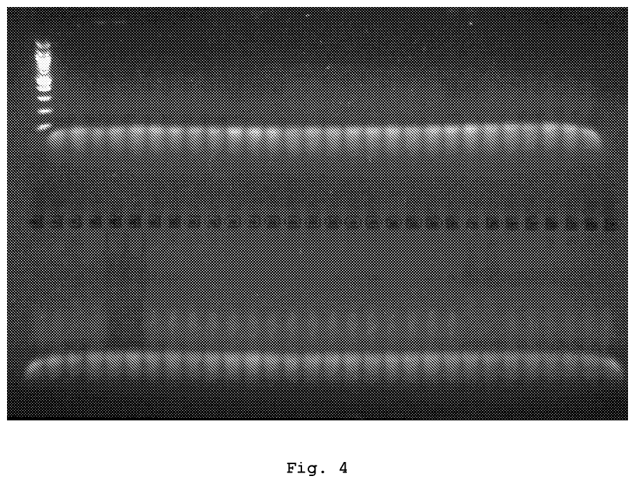

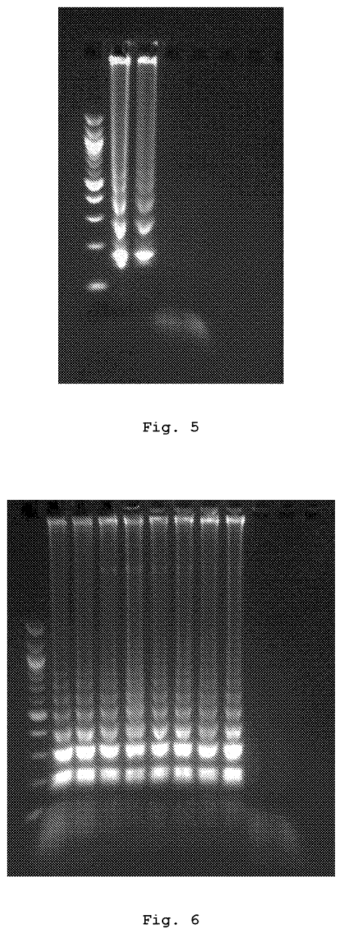

The sensitivity was determined by assaying serial dilutions of the Neisseria meningitidis Quantitative DNA (ATCCR 700532DQ™) standard with a minimum amount of 5 copies of bacteria per reaction mixture, where the product amplification was measured in real time—FIG. 2 (Real-Time LAMP for serial dilutions) along with recording the dissociation temperature of 88.5° C. (FIG. 3).

The time required to detect the emitted fluorescence for individual samples is shown in Table 1.

The characterized primers allow for the detection of Neisseria meningitidis bacteria by detecting the FrpA gene fragment at a minimum number of 5 copies/reaction mixture.

TABLE 1
Time required to detect fluorescence for each
dilution of the Neisseria meningitidis Quantitative
DNA (ATCC ® 700532DQ ™) standard.

| Sample | Time to exceed the baseline fluorescence [min] |
|---|---|
| NTC | Indefinite |
| NM 5 copies | 24.87 |
| NM 10 copies | 19.52 |
| NM 20 copies | 19.58 |
| NM 25 copies | 18.67 |
| NM 50 copies | 19.13 |
| NM 100 copies | 17.91 |
| NM 1000 copies | 15.52 |

### Example 8. Specificity of the Method

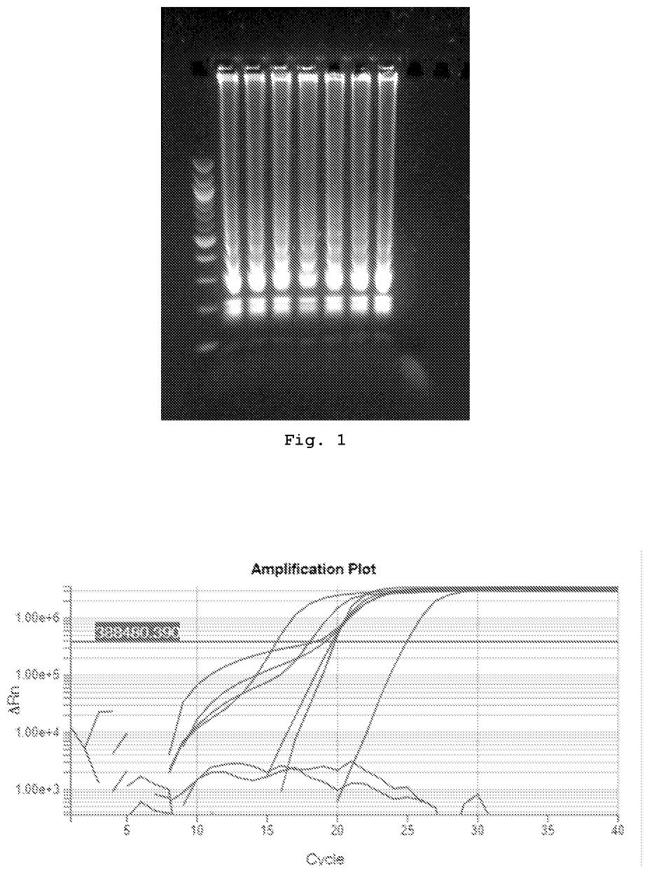

The superiority of the amplification method and the oligonucleotides described in this specification over the tests based on the Real-Time LAMP technology is due to the much higher sensitivity, which is shown in FIG. 1, and the reduction of the analysis time shown in FIG. 2.

1. A set of primers for amplifying the nucleotide sequence of the FrpA gene of Neisseria meningitidis, characterized in that it comprises a set of internal primers with the following nucleotide sequences a) and b), as well as a set of external primers comprising the following nucleotide sequences c) and d):
a) 5′ CGAGCGTATCATTGCCATTGCC 3′ (SEQ ID NO: 3) or a sequence at least 90% identical to SEQ ID NO: 3, linked from the 3′ end, preferably by a TTTT bridge, to the sequence 5′ CGGGGATGACCTGCTGAA 3′-(SEQ ID NO: 4) or a sequence at least 90% identical to SEQ ID NO: 4;
b) 5′ ACGACGCCCTGTACGGCTATA 3′-(SEQ ID NO: 5) or a sequence at least 90% identical to SEQ ID NO: 5, linked from the 3′ end, preferably by a TTTT bridge, to the sequence 5′ TACCGTCTTCGCCGTTCAA 3′-(SEQ ID NO: 6) or a sequence at least 90% identical to SEQ ID NO: 6;
c) 5′ GGGCGATGACTATCTGTACG 3′ nucleic sequence of (SEQ ID NO: 1) or a sequence at least 90% identical to SEQ ID NO: 1, and
d) 5′ CACCGCCGATTAGAGTGTC 3′ nucleic sequence (SEQ ID NO: 2) or a sequence at least 90% identical to SEQ ID NO: 2.

2. The set of primers of claim 1, characterized in that it comprises a set of loop primer sequences comprising nucleic sequences contained in or complementary to the Neisseria meningitidis FrpA gene 5′ TACTGTCGTTGCCTGCATCACC 3′ (SEQ ID NO: 7) or a sequence at least 90% identical to SEQ ID NO: 7 and 5′ GGTAACGATGTACTGAATGGTGG 3′ (SEQ ID NO: 8) or a sequence at least 90% identical to SEQ ID NO: 8.

3. A method of detecting Neisseria meningitidis bacteria, characterized in that a selected region of the nucleic sequence of the bacterial genome is amplified using the set of primers as defined in claim 1, the amplification method being the LAMP method.

4. The method of detecting bacteria of claim 3, characterized in that the amplification is carried out with a temperature profile of:
68° C., 40 min

5. The method of claim 4, characterized in that an end-point reaction is carried out with a temperature profile of 80° C., 5 min. after the amplification stage.

6. A method for detecting infection caused by the Neisseria meningitidis bacterium, characterized in that it comprises the detection method as defined in claim 3.

7. A kit for detecting infection caused by the Neisseria meningitidis bacterium, characterized in that it comprises the set of primers as defined in claim 1.

8. The kit for detecting infection of claim 7, characterized in that it comprises 5.0 μl of WarmStart LAMP Master Mix (NEB).

9. The kit for detecting infection of claim 7, wherein the primers have the following concentrations:
primer c) at 0.13 μM,
primer d) at 0.13 μM,
primer b) at 1.06 μM, and
primer a) at 1.06 μM.

10. A method of detecting Neisseria meningitidis bacteria, characterized in that a selected region of the nucleic sequence of the bacterial genome is amplified using the set of primers as defined in claim 2, the amplification method being the LAMP method.

11. The method of detecting bacteria of claim 10, characterized in that the amplification is carried out with a temperature profile of:
65.5° C., 40 min.

12. The method of claim 11, characterized in that the end-point reaction is carried out with a temperature profile of 80° C., for additional 5 min.

13. A method for the detection of a Neisseria meningitidis bacterium infection, characterized in that it comprises the detection method of claim 10.

14. A kit for the detection of Neisseria meningitidis bacterium infection, characterized in that it comprises a set of primers as defined in claim 2.

15. The infection detection kit of claim 14, characterized in that it comprises 5.0 μl of WarmStart LAMP 2× Master Mix (NEB).

16. The infection detection kit of claim 14, wherein the primers have the following concentrations:
primer c) at 0.13 μM,
primer d) at 0.13 μM,
primer b) at 1.06 μM,
primer a) at 1.06 μM, and
each of the loop primers at 0.26 μM.

17. The infection detection kit of claim 9, comprising D-(+)-Trehalose dihydrate.

18. The infection detection kit of claim 9, comprising a fluorescent marker interacting with double-stranded DNA.

19. The infection detection kit of claim 14, comprising D-(+)-Trehalose dihydrate.

20. The infection detection kit of claim 14, comprising a fluorescent marker interacting with double-stranded DNA.


SEQUENCE LISTING (9 sequences)

SEQ ID NO: 1 - DNA, 20 bp, synthetic construct
  misc_feature 1..20: note = primer
  source 1..20: mol_type = other DNA
gggcgatgactatctgtacg

SEQ ID NO: 2 - DNA, 19 bp, synthetic construct
  misc_feature 1..19: note = primer
  source 1..19: mol_type = other DNA
caccgccgattagagtgtc

SEQ ID NO: 3 - DNA, 22 bp, synthetic construct
  misc_feature 1..22: note = primer
  source 1..22: mol_type = other DNA
cgagcgtatcattgccattgcc

SEQ ID NO: 4 - DNA, 18 bp, synthetic construct
  misc_feature 1..18: note = primer
  source 1..18: mol_type = other DNA
cggggatgacctgctgaa

SEQ ID NO: 5 - DNA, 21 bp, synthetic construct
  misc_feature 1..21: note = primer
  source 1..21: mol_type = other DNA
acgacgccctgtacggctata

SEQ ID NO: 6 - DNA, 19 bp, synthetic construct
  misc_feature 1..19: note = primer
  source 1..19: mol_type = other DNA
taccgtcttcgccgttcaa

SEQ ID NO: 7 - DNA, 22 bp, synthetic construct
  misc_feature 1..22: note = primer
  source 1..22: mol_type = other DNA
tactgtcgttgcctgcatcacc

SEQ ID NO: 8 - DNA, 23 bp, synthetic construct
  misc_feature 1..23: note = primer
  source 1..23: mol_type = other DNA
ggtaacgatgtactgaatggtgg

SEQ ID NO: 9 - DNA, 309 bp, Neisseria meningitidis
  misc_feature 1..309: note = gen
  source 1..309: mol_type = genomic DNA
ggcggattgggcgatgactatctgtacggtgccgacggggatgacctgctgaatggtgatgcaggcaacgacagtatctacagtggcaatggcaatgatacgctcgatggaggagaaggcaacgacgccctgtacggctataatggtaacgatgtactgaatggtggcgaaggcaatgatcatttgaacggcgaagacggtaacgacactctaatcggcggtgcaggcaatgattacttggagggcggcagcggttcggatacttatgtcttcggcgaaggcttcggtcaggatacggtctataattac